(19) United States
(12) Patent Application Publication
(10) Pub. No.: US 2024/0102059 A1
(43) Pub. Date: Mar. 28, 2024

(54) METHOD FOR MANUFACTURING TRIACYLGLYCEROLS ENRICHED EITHER IN PALMITIC ACID AT SN-2 POSITION OR OLEIC ACID AT SN-2 POSITION

(71) Applicant: SOCIETE DES PRODUITS NESTLE S.A., Vevey (CH)

(72) Inventor: AMAURY PATIN, Lausanne 26 (CH)

(21) Appl. No.: 18/263,763

(22) PCT Filed: Feb. 3, 2022
(86) PCT No.: PCT/EP2022/052549

§ 371 Date: Aug. 1, 2023

(30) Foreign Application Priority Data
Feb. 9, 2021 (EP) 21156115.4

(51) Int. Cl. C12P 7/62; C12N 9/20; C12P 7/6454; C12P 7/6458

(52) U.S. Cl. CPC C12P 7/62; C12N 9/20; C12P 7/6454; C12P 7/6458; C12Y 301/01003

(57) ABSTRACT

The present invention concerns an enzymatic process for the preparation of an ingredient comprising triacylglycerols enriched either in palmitic acid at sn-2 position or in oleic acid at sn-2 position.

### FIELD OF THE INVENTION

The present invention concerns an enzymatic process for the preparation of an ingredient comprising triacylglycerols enriched either in palmitic acid at sn-2 position or in oleic acid at sn-2 position.

### BACKGROUND OF THE INVENTION

Triacylglycerols (TAG) are the major lipids found in human milk at about 39 g/L and they present a specific regiospecific distribution of fatty acids. The regio-specific distribution of TAG contributes to the nutritional benefits of human milk such as to fatty acid and calcium absorption and their related benefits such as gut comfort. Infant formula (IF) ingredient design is generally aimed at structural and functional homology with respect to human milk composition and benefits.

Currently, OPO (1,3-Dioleo-2-palmitin) enriched ingredients are already incorporated into some IF. They are produced using enzymatic reactions (for example Betapol® or Infat®) but the OPO content in these ingredients ranges only from 20 to 28% w/w of total TAG, the rest being other TAG (for example POO (2,3-Dioleo-1-palmitin), which may range from 5 to 8% w/w of total TAG). The low OPO content of these ingredients coupled with presence of other TAG represents a limit for their use in the preparation of IF having a fat portion reproducing as close as possible the fat content of human breast milk.

Other OPO synthesis are also known on lab scale using enzymatic reactions. These reactions however are either not possible to scale up at an industrial level (due to the use of large volumes of organic solvents and of complex and costly purification steps to yield the desired OPO content and/or selectivity over other TAG) or they are not capable to deliver an ingredient with desired OPO content and/or selectivity over other TAG.

As an alternative to produce structured lipid with high sn-2 palmitic acid content, literature described the enzymatic two-step approach via the alcoholysis of triglycerides into 2-monoglyceride intermediate (Schmid et al, 1999) and its sub-sequent esterification with FFA (free fatty acids), which could offer higher reaction control, purity and yield. However, this two-step process described in the literature required the use of solvents as well as costly purification steps. For an enzymatic 2-step process to become economically viable and industrially applicable, cost and starting material composition should be taken into consideration, solvent use needs to be reduced or removed and purifications must be simplified, while maintaining high purity and high selectivity of the OPO ingredient obtained, for example a minimum of 50% overall OPO purity and overall a minimum of 70% of the total palmitic acid (PA) at sn-2 position.

The Applicant of the present application has already addressed the above mentioned problem and provided a solution thereto as described in European patent application EP20168959.3 from the same applicant (still unpublished) by providing a simplified, solvent-free, two-step enzymatic method for producing an OPO enriched ingredient with an overall content of palmitic acid at position sn-2 larger than 70%, for example 75%. This simplified enzymatic process concept offers an economically viable route towards OPO enriched ingredient production.

One drawback of the above-mentioned process described in European patent application EP20168959.3 is anyway that of generating high amount of fatty acid butyl esters as by-products which are not further used in the process and which also need to be disposed of. Such by-product is, for example, butyl palmitate.

There remains thus a need to provide a process for the preparation of an OPO ingredient with an OPO purity of at least 50 g/100 g of the ingredient and with an overall content of palmitic acid at sn-2 position which is equal or higher than 70% of total palmitic content, such process being economically viable and industrially applicable and whose by-products may be employed differently and do not need to be disposed of.

### SUMMARY OF THE INVENTION

An object of the present invention is to improve the state of the art and to provide an improved solution to overcome at least some of the inconveniences described above. The object of the present invention is achieved by the subject matter of the independent claims. The dependent claims further develop the idea of the present invention.

Accordingly, the present invention provides in a first aspect a process for the preparation of ingredients comprising triacylglycerols enriched either in palmitic acid at sn-2 position or in oleic acid at sn-2 position as described in the attached claims.

The inventors have surprisingly found that the 2-step process involving enzymatic reaction for producing OPO in high quality, as described in the European patent application EP20168959.3, can be used to produce POP (2-Oleo-1,3-dipalmitin), using the same enzyme, similar processing steps with adapted conditions and changing the raw materials (i.e. TAG and FFA). The inventors have then found that it is advantageously possible to use the by-product produced in one process, for example from the process for producing OPO, as a reactant material in the other process, for example in the process for producing POP, and vice-versa. Therefore, by-product generated in the alcoholysis step of the process for producing POP, i.e. oleate ester, for example butyl oleate, can be used as reactant in the esterification step of the process to produce OPO. Vice-versa, the by-product generated in the alcoholysis step of the process for producing OPO, i.e. palmitate ester, for example butyl palmitate, can be used in the esterification step of the process to produce POP.

The process according to the present invention has then the advantage that the by-products generated in the 2-step processes involving the enzymatic reaction used to produce either OPO or POP can be recovered and re-used as reactants. Therefore, the by-products that are usually considered as waste are recovered and recycled as reactants, thereby optimizing resource efficiency across the chemical value chain and enabling a closed-loop, waste free chemical reaction. The inventors have then found an economically viable and cost-effective route towards OPO enriched ingredient production while allowing for concomitant production of a POP enriched ingredient which may be of use in the preparation of cocoa butter equivalents.

The process according to the present invention has the additional advantage that the addition of a controlled mixture of FFA into the process can tailor-made the final TAG composition to either match the human milk composition (e.g. OPO/OPL (1-Oleo-2-palmito-3-linolein) at different ratio) or match the cocoa butter composition (POP, SOP (2-Oleo-1,3-distearin) and SOS (1-Oleo-1,3-distearin) also at different ratio).

In a second aspect, the present invention relates to a process for the preparation of triacylglycerols enriched in oleic acid at sn-2 position as described in the attached claims. This simplified enzymatic process concept offers an economically viable route towards ingredients comprising triacylglycerols enriched in oleic acid at sn-2 position that are abundant in cocoa butter and cocoa butter equivalents.

In a further aspect, the invention relates to the use of a process for the preparation of triacylglycerols enriched in oleic acid at sn-2 position in another process for the preparation of ingredients enriched in triacylglycerols enriched either in palmitic acid at sn-2 position or in oleic acid at sn-2 position as described in the attached claims.

### BRIEF DESCRIPTION OF THE DRAWINGS

Additional features and advantages of the present invention are described in, and will be apparent from, the description of the presently preferred embodiments which are set out below with reference to the drawings in which:

FIG. 1 shows a schematic representation on the overall process according to some embodiments of the present invention. FIG. 1A shows the process according to the present invention wherein by-products generated in the alcoholysis steps are re-used as reactants in the esterification steps to produce either OPO or POP. FIG. 1B shows the process according to the present invention wherein the esterification steps are performed in the presence of by-products generated in the alcoholysis steps together with FFA to produce TAG composition better matching the human breast milk composition and the cocoa butter equivalent composition, respectively. In FIG. 1, the following acronyms are used: PPP indicates tripalmitin ingredient from, for instance, palm oil fraction high in tripalmitin; OPO indicates a triglyceride composed of 2 oleic and 1 palmitic acids (palmitic in position sn-2); OOO indicates triolein from, for instance, High Oleic Sunflower oil (HOSFO); POP indicates triglyceride composed of 1 oleic and 2 palmitic acids (oleic in position sn-2); Enzyme indicates any sn-1,3 selective lipases, for instance, Novozymes Lipozyme® TL IM, immobilized enzymes produced from Thermomyces lanuginosus (fungus).

FIG. 2 shows a schematic representation on the processes according to some embodiments of the present invention FIG. 2A shows a schematic representation of the process for the preparation of ingredients comprising triacylglycerols enriched either in palmitic acid at sn-2 position or in oleic acid at sn-2 position according to one embodiment of the present invention. FIG. 2B shows a schematic representation for the preparation of triacylglycerols enriched in oleic acid at sn-2 position according to one embodiment of the present invention.

FIG. 3 shows a schematic representation of the process described in the patent application EP20168959.3 from the same applicant and still unpublished.

FIG. 4 shows results of Example 1 and reports Yields of 2-monopalmitin over the reaction time for alcoholysis reaction using lipases 435 and TL IM with different alcohols. Yields are calculated as mol 2-monopalmitin/mol initial tripalmitin.

FIG. 5 shows Conversion profile for isopropanolysis of tripalmitin catalysed by Lipozyme TL IM as described in Example 1.

FIG. 6 shows each quantified species in the reaction mixture of Example 2 as a percentage of total quantified palmitic acid containing compounds.

FIG. 7 shows content of alcoholysis product compared to the precipitate from fractionation of the same mix (Example 4).

FIG. 8 shows variations of the species in the reaction mixture of solvent free esterification of 2-monoplamitic product (Example 5) based on gas chromatography (GC).

FIG. 9 shows the fatty acid distribution in the final TAG mixture for Example 5 determined by LC-MC analysis.

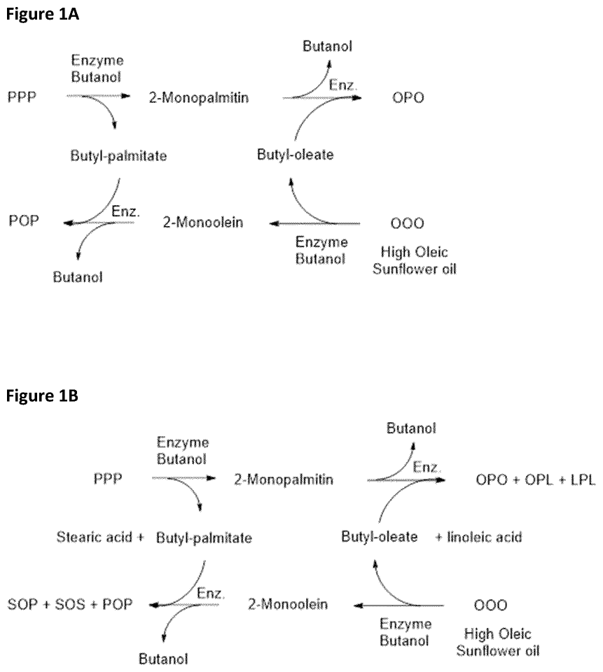

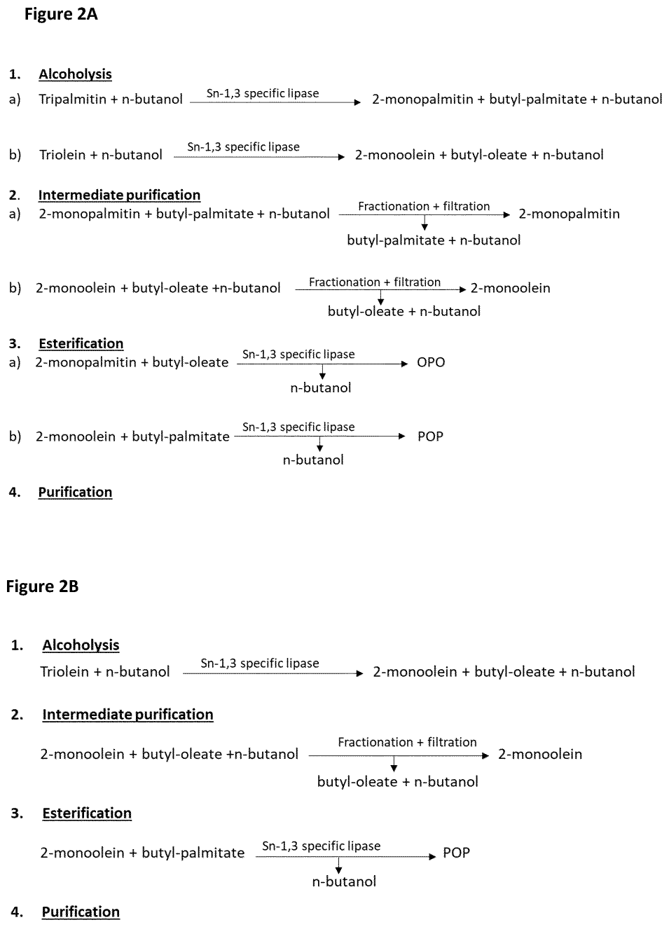

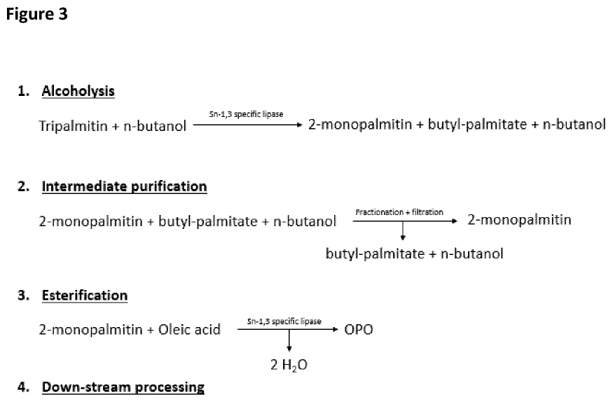

### DETAILED DESCRIPTION OF THE INVENTION

### Definitions

Within the context of the present invention the term “OPO” refers to 1,3-Dioleo-2-palmitin and/or 2-(palmitoyloxy)propane-1,3-diyl dioleate and/or (2-(Palmitoyloxy)-1,3-propanediyl (9Z,9′Z)bis(-9-octadecenoate) (CAS number: 1716-07-0).

Within the context of the present invention the term “POP” refers to 2-Oleo-1,3-dipalmitin and/or 1,3-Dihexadecanoyl-2-(9i-octadecenoyl)glycerol (CAS number: 2190-25-2).

Within the context of the present invention the term “OOO” refers to triolein and/or 9-Octadecenoic acid (9Z)-, 1,1′,1″-(1,2,3-propanetriyl) ester (CAS number: 122-32-7).

Within the context of the present invention the term “PPP” refers to tripalmitin and/or Hexadecanoic acid, 1,1′,1″-(1,2,3-propanetriyl) ester (CAS number: 555-44-2).

Within the context of the present invention the term “OPL” refers to 1-Oleo-2-palmito-3-linolein (CAS number: 2534-97-6).

Within the context of the present invention the term “SOP” refers to the racemic 2-Oleo-3-palmito-1-stearin and/or 2-Oleo-1-palmito-3-stearin (CAS number: 2190-27-4).

Within the context of the present invention the term “SOS” refers to 2-Oleo-1,3-distearin (CAS number: 2846-04-0).

Within the context of the present invention the term “POO” refers to both 3-(Palmitoyloxy)-1,2-propanediyl (9Z,9′Z)bis(-9-octadecenoate), (OOP, CAS number: 14960-35-1), and/or 1-(Palmitoyloxy)-2,3-propanediyl (9Z,9′Z)bis(-9-octadecenoate), (POO, CAS number: 14863-26-4). It is to be noted that when reference is made to amounts of “POO”, this also includes amounts of OOP present in the ingredient.

Within the context of the present invention, the term “OPO Ingredient” or “OPO enriched Ingredient” or “1,3-Dioleo-2-palmitin ingredient” identifies an edible ingredient comprising OPO with purity higher than 50 g/100 g of the ingredient. In one embodiment of the present invention, the OPO ingredient prepared according to the process also has a content of palmitic acid in sn-2 position which is equal or higher than 70% of total palmitic content.

Within the context of the present invention, the term “POP Ingredient” or “POP enriched Ingredient” or “2-Oleo-1,3-dipalmitin ingredient” identifies an edible ingredient comprising POP with purity higher than 50 g/100 g of the ingredient. In one embodiment of the present invention, the POP ingredient prepared according to the process also has a content of oleic acid at sn-2 position which is equal or higher than 70% of total oleic content.

Within the context of the present invention, the term “TAG” means triacylglycerols or triglycerides.

Within the context of the present invention, the term “triacylglycerol(s) enriched in palmitic acid at sn-2 position” means triacylglycerol(s) and/or triacylglycerol ingredient wherein a proportion higher than 70% of palmitic acid residues are at sn-2 position in the triacylglycerol backbone. In one embodiment, the triacylglycerols enriched in palmitic acid at sn-2 position have a proportion higher than 75% of palmitic acid residues at sn-2 positions in the triacylglycerol backbone. In another embodiment, the triacylglycerols enriched in palmitic acid at sn-2 position have a proportion higher than 80% of palmitic acid residues at sn-2 positions in the triacylglycerol backbone. In some embodiments, the triacylglycerols enriched in palmitic acid at sn-2 position is a palm oil fraction enriched in triacylglycerol containing palmitic acid, such as for example palm stearin with IV (iodine value) below 10 with tripalmitin content >60% w/w and wherein the proportion of sn-2 position in the triglyceride backbone occupied by palmitic acid residues is higher than 70%, for example higher than 75% or higher than 80% .

Within the context of the present invention, the term “triacylglycerols enriched in oleic acid at sn-2 position” means triacylglycerols and/or triacylglycerol ingredient wherein a proportion higher that 70% of oleic acid residues are at sn-2 position in the triacylglycerol backbone. In one embodiment, the triacylglycerols enriched in oleic acid at sn-2 position have a proportion higher than 75% of oleic acid residues at sn-2 position in the triacylglycerol backbone. In another embodiment, the triacylglycerols enriched in oleic acid at sn-2 position have a proportion higher than 80% of oleic acid residues at sn-2 position in the triacylglycerol backbone.

Within the context of the present invention, the term “circular process” or “circular method” means a combination of two processes each resulting in distinct end products and generating by-products, and wherein the by-products generated in one process is used as a reactant in the second process and vice-versa. Therefore, the by-products that are usually considered as waste are recovered and recycled as reactants, thereby optimizing resource efficiency across the chemical value chain and enabling a closed-loop, waste free chemical reaction.

Within the context of the present invention, the term “by-product” means a secondary product produced during the preparation of the principal product in a reaction or process.

Within the context of the present invention the term “cocoa butter equivalent” (CBE) means a cocoa butter substitute for chocolate applications. Cocoa butter equivalents are usually made from vegetable fat, for example formulated from palm oil, shea butter, sal fat or illipe butter, by way of fractionation process and resembles cocoa butter in both physical and chemical properties due to the similarity in the TAG composition with the same three main TAG found in both: POP, SOP and SOS.

Within the context of the present invention, the term “alcoholysis” means the transesterification reaction of fatty acids present in a triglyceride with an alcohol (methanol, ethanol, butanol . . . ) by the action of a selective sn-1,3 lipase enzyme. This reaction leads to the formation of monoglycerides and fatty acid esters of the respective alcohol and fatty acids.

Within the context of the present invention, the term “lipase” or “sn-1,3 lipase” means a hydrolytic enzyme that acts on ester bonds (EC 3.1) and belongs to the class of carboxylic-ester hydrolases (EC 3.1.1), and more specifically possesses a high regio-selectivity for hydrolyzing the Sn-1 and Sn-3 ester bond in a triglyceride backbone. Lipases with high 1,3-selectivity can be sourced, for example, from Candidata antarctica (lipase B), Thermomyces lanuginosus, Rhizomucor miehei, R. oryza, Rhizopus delemar, etc.

Non limiting examples of sn-1,3 lipase in immobilized form are: lipase from Thermomyces lanuginosis adsorbed on silica (e.g., Lipozyme TL IM, Novozymes), lipase B from Candida antarctica adsorbed on methacrylate/divinylbenzene copolymer (e.g. Lipozyme 435, Novozymes), lipase from Rhizomucor miehei attached via ion exchange on styrene/DVB polymer (e.g., Novozym® 40086, Novozymes) or via hydrophobic interaction onto macroporous polypropylene (Accurel EP 100).

Within the context of the present invention, the term “deodorization” means a steam distillation process in which steam is injected into an oil under conditions of high temperature (typically>200° C.) and high vacuum (typically<20 mBar) to remove volatile components like free fatty acids (FFA), fatty acid esters, mono- and diglycerides and to obtain an odourless oil composed of TAG.

Within the context of the present invention, the term “fractionation” means a separation process in which a certain quantity of a mixture (solid, liquid, suspension) is separated into fractions during a phase transition. These fractions vary in composition thus usually allowing enrichment of a species in one of the fractions and its subsequent separation and/or purification.

Within the context of the present invention, the term “selective precipitation” or “selective crystallization” indicates a separation and/or purification technique whereby the creation of one or several specific precipitates (solids) occur from a solution containing other potential precipitates by means of adapting the temperature of the precipitation. For example, the species having a melting point above the temperature of the precipitation process will not form a precipitate under those conditions.

In one embodiment of the present invention, the selective precipitation results in crystallization of the desired product.

Within the context of the present invention, the term “immobilized form” means that the lipase enzyme is attached either covalently or non-covalently (e.g. adsorbed) to a solid carrier material. Non limiting examples of suitable carriers are:macroporous hydrophobic supports for covalent attachment made of methacrylate resins with, for example, epoxy, butyl or amino groups together with a suitable linker molecule (e.g. glutaraldehyde); for non-covalent immobilization through hydrophobic interactions via macroporous carriers made of, e.g., polystyrenic adsorbent, octadecyl methacrylate, polypropylene, non-compressible silica gel; for non-covalently adsorption via ionic interactions ionic exchange resins are used, e.g., polystyrenic ion exchange resin or silica.

### Process for the Preparation of Ingredients Comprising Triacylglycerols Enriched in Either Palmitic Acid at sn-2 Position or in Oleic Acid at sn-2 Position

The inventors have found a circular process for the preparation of ingredients comprising triacylglycerols enriched either in palmitic acid at sn-2 position or in oleic acid at sn-2 position, this circular process combining two processes each generating distinct by-products and resulting in distinct end products. The two processes are temporally and/or spatially separated. It means that one process may be performed in parallel, in a separated manufacturing line for example, or after the completion of the other, either in the same manufacturing line or in a separate line. One of the enzymatic processes combined in the circular process results in an ingredient comprising triacylglycerols enriched in palmitic acid at sn-2 position that are abundant in human breast milks. The second enzymatic process combined in the circular process results in an ingredient comprising triacylglycerols enriched in oleic acid at sn-2 position that are abundant in cocoa butter and cocoa butter equivalents. These two enzymatic processes share common steps, i.e a first alcoholysis step (steps a)i) and steps b)i)), followed by an intermediate purification step (step a)ii) and step b)ii)) and a solvent-free esterification step (step c)i) and step c)ii)). A purification step (step a)iv) and step b)iv)) can be performed after the esterification step.

The process according to the present invention has then the advantage that the by-products generated in the 2-step processes involving the enzymatic reaction used to produce either OPO or POP can be recovered and re-used as reactants. In some embodiments of the present invention, all or at least a portion of the palmitate ester generated in step a)i) and of the oleate ester generated in step b)i) is recycled in the respective processes after removal of the remaining alcohol by evaporation.

This circular process has the advantage of re-using by-products generated during these two processes. Therefore, the by-products that are usually considered as waste are recovered and recycled as reactants, thereby optimizing resource efficiency across the chemical value chain and enabling a closed-loop, waste free chemical reaction.

### Alcoholysis [steps a)i) and b)i)]

The process of the present invention comprises a process comprising the step of subjecting tripalmitin and/or triglycerides enriched in tripalmitin to an alcoholysis step in the presence of an immobilized lipase and of primary or secondary alcohol of chain length C3-C5 to give a product mixture comprising 2-monopalmitin and palmitate ester as a by-product.

In some embodiments of the present invention, the starting material for alcoholysis step a)i) is tripalmitin. In other embodiments of the present invention, the starting material for alcoholysis step a)i) is a triacylglycerol mixture enriched in tripalmitin, such as for example a palm oil fraction enriched in palmitic acid, for example palm stearin with IV (iodine value) below 10.

A challenge with selective alcoholysis of tripalmitin and/or triacylglycerols enriched in tripalmitin into 2-monopalmitin is the high melting point of tripalmitin (about 65° C.). Chemical alcoholysis is non-specific and can thus not be used to produce 2-monopalmitin. On the contrary, enzymatic alcoholysis can lead to a highly selective alcoholysis at the sn-1,3 positions making high purity synthesis of 2-monopalmitin possible. The problem of using enzymes is the relatively poor thermostability of most of the commercial enzymes and results in lipase inactivation when reactions are performed at above 50° C. To minimize lipase inactivation and achieve full solubilization of the substrate (e.g. tripalmitin) at lower temperatures (<50° C.), organic solvents, most commonly acetone, n-hexane, or MTBE, are typically used. However, the use of solvents for industrial application increases the process complexity and operations (solvent removal and handling, safety), and thus drive the process costs (of solvent, larger reaction volumes and thus equipment/reactors) as well as pose an environmental burden (solvent recycling).

The process of the present invention also comprises performing another process that is temporally and/or spatially separated from the other process comprising the step of subjecting triolein and/or triacylglycerols enriched in triolein to an alcoholysis step in the presence of an immobilized lipase and of primary or secondary alcohol of chain length C3-C5 to give a product mixture comprising 2-monoolein and oleate ester as a by-product.

In some embodiments of the present invention, the starting material for alcoholysis step b)i) is triolein. In other embodiments of the present invention, the starting material for alcoholysis step b)i) is a triacylglycerol mixture enriched in triolein, such as for example high oleic sunflower oil.

In some embodiments of the present invention, the alcoholysis steps a)i) and b)i) are performed in the presence of n-butanol, n-pentanol, isopropanol or mixtures thereof.

In some embodiments, the primary or secondary alcohol of chain length C3-C5 is selected from the list consisting of n-butanol, n-pentanol, isopropanol and mixture thereof.

In the context of the present invention, switching from the commonly used alcohols, methanol and ethanol, to n-butanol supplied at high molar ratio (6 to 15 equivalents) has surprisingly allowed the substrate to be solubilized at 50° C. without deactivating the enzyme, producing 2-monopalmitin with 90% purity.

In some embodiments of the present invention, the alcoholysis steps a)i) and b)i) are performed in the presence of n-butanol. The n-butanol may be in excess.

In some embodiments, alcoholysis steps a)i) and b)i) are performed in the presence of an sn-1,3 lipase selected in the group consisting of: lipase from Thermomyces lanuginosis adsorbed on silica (e.g., Lipozyme TL IM, Novozymes), lipase B from Candida antarctica adsorbed on methacrylate/divinylbenzene copolymer (e.g. Lipozyme 435, Novozymes) and lipase from Rhizomucor miehei attached via ion exchange on styrene/DVB polymer (e.g., Novozym® 40086, Novozymes) or via hydrophobic interaction onto macroporous polypropylene (Accurel EP 100). In other embodiments, alcoholysis steps a)i) and b)i) are performed in the presence of an sn-1,3 lipase, for example a lipase from Thermomyces lanuginosis, adsorbed on silica (e.g., Lipozyme TL IM, Novozymes).

In another embodiment, tripalmiin and/or triacylglycerols enriched in tripalmitin is subjected to an alcoholysis step a)i) performed in the presence of an sn-1,3 lipase and of n-butanol to give a product mixture comprising 2-monopalmitin and butyl palmitate as a by-product.

In some embodiments, triolein and/or triacylglycerols enriched in triolein is subjected to an alcoholysis step b)i) performed in the presence of an sn-1,3 lipase and of n-butanol to give a product mixture comprising 2-monoolein and butyl oleate as a by-product.

In alcoholysis steps a)i) and b)i), immobilized enzyme preparation allows to properly disperse the lipase in non-aqueous media, such as fats and solvents, and enables the recovery and reuse making the process more cost efficient.

In some embodiments of the present invention, the alcoholysis steps a)i) and b)i) are performed in the presence of n-butanol and of a sn-1,3 lipase, adsorbed on silica gel carrier. In other embodiments of the present invention, the alcoholysis steps a)i) and b)i) are performed in the presence of n-butanol and of a lipase from Thermomyces lanuginosis adsorbed on silica gel carrier.

By using n-butanol in alcoholysis steps a)i) and b)i), the reaction proceeded without any solvent at temperature ranging from 50 to 65° C. Butanol acts as both substrate and solubilization agent for the triglycerides, thereby, enabling a solvent-free reaction, high conversion yield (excess) and lipase activity.

In some embodiments of the present invention, the alcoholysis steps a)i) and b)i) are performed at a temperature ranging from 40 to 70° C., for example at a temperature ranging from 45 to 55° C.

In some embodiments of the present invention, the alcoholysis step a)i) gives a product mixture comprising 2-monopalmitin and palmitate ester as a by-product. In other embodiments of the present invention, the alcoholysis step b)i) gives a product mixture comprising 2 -monooleate and oleate ester as a by-product. When the alcoholysis steps a)i) and b)i) are performed in the presence of n-butanol, it gives a product mixture comprising 2-monopalmitin and butyl palmitate on one hand and a product mixture comprising of 2-monoolein and butyl oleate on the other hand. Accordingly, in some embodiments of the present invention, the by-product generated in alcoholysis step a)i) is butyl palmitate and the by-product generated in step b)i) is butyl oleate.

Accordingly, alcoholysis step as described in the present invention provides several advantages to the process according to the present invention, for example:
solvent-free reaction allows smaller reactor volumes (increased volumetric productivity), lowered process costs and omits safety handling aspects, removal and recycling of the solvent (solvent removal is especially important for an ingredient aimed at infant nutrition);
Immobilized lipases, such as Lipozyme TL IM (Novozymes), are commercially available lipases accessible at industrial scale;
The by-products generated in the alcoholysis steps can be re-used in subsequent esterification reactions, thereby

### Intermediate Purification [steps a)ii) and b)ii)]

The two-step enzymatic transesterification process according to the present invention is more complex than conventional methods of producing OPO or POP, e.g. single step acidolysis, yet, the moderate increase in complexity enables to improve the quality in the final product significantly, i.e. higher sn-2 palmitate content or higher sn-2 oleate, respectively, making it more attractive for use in IF and in cocoa butter equivalents, respectively.

A two-step process requires the purification of the intermediate and it is important that the increase in quality is not offset by increase in cost potentially deriving from intermediate purification [steps a)ii) and b)ii)].

Current technologies for intermediate purification include molecular distillation, solvent crystallization, and chromatography but all these three methods are too costly for the targeted application and would benefit from improvement/simplification. For example, solvent fractionation methods typically require solvent use and low temperatures (<−10° C.).

According to the process of the present invention, the intermediate purification steps a)ii) and b)ii) may be performed by selective crystallization of 2-monopalmitin or 2-monooleate, respectively.

Accordingly, the process of the present invention comprises a process comprising the step of purifying the product mixture comprising 2-monopalmitin obtained in alcoholysis step a)i) by fractionation process via selective crystallization of 2-monopalmitin and subsequent removal of the remaining liquid fraction comprising palmitate ester and the remaining liquid fraction comprising palmitate ester and the remaining alcohol, for example by filtration of by centrifugation. The side product to be removed in this purification step is the product of the reaction of the alcohol (methanol, ethanol, butanol . . . ) with the fatty acids present in position 1,3 (mainly palmitic acid). The resulting esters have different melting points depending on the alcohol used. In particular, butyl palmitate has a lower melting point (17° C.) than methyl and ethyl palmitic esters (30° C. and 24° C. respectively), providing a larger difference in melting point between 2-monopalmitin (60° C.) and the side products to be removed. This higher difference is beneficial for the separation process. Such side products including the excess of alcohol used in the alcoholysis can be effectively removed after the alcoholysis step a)i) by fractionation of the crude mixture containing 2-monopalmitin, butanol and butyl palmitate at temperatures ranging from 0 to 15° C., whereby the 2-monopalmitin undergoes selective crystallization and the side products remain in the liquid state and can be filtered off, for example.

Accordingly, fractionation temperatures above 0° C. of the crude mixtures and no addition of solvents allows for a simple and cheap purification step of 2-monopalmitin.

Using solvent-free fractionation, the selective crystallization of the target product (2-monopalmitin) can be performed at higher temperature and there is no need to perform a step for solvent removal by distillation.

In some embodiments of the present invention, intermediate purification step a)ii) is performed by decreasing the temperature of the product mixture comprising 2-monopalmitin obtained in step a)i) to a temperature ranging from 0° C. to 15° C., for example to a temperature ranging from 5° C. to 10° C. or to a temperature ranging from 6° C. to 8° C. to allow fractionation via selective crystallization of 2-monopalmitin and by removing the remaining liquid fraction, for example by filtration or by centrifugation.

In some embodiments of the present invention, intermediate purification step a)ii) is performed by decreasing the temperature of the product mixture comprising 2-monopalmitate obtained in step a)i) to a temperature ranging from 0 to 15° C. to allow fractionation via selective precipitation of 2-monopalmitin and by removing the remaining liquid fraction, for example by filtration or centrifugation.

The process of the present invention also comprises performing another process that is temporally and/or spatially separated from the other process comprising the step of purifying the product mixture comprising 2-monoolein obtained in alcoholysis step b)i) by fractionation process via selective crystallization of 2-monoolein and subsequent removal of the remaining liquid fraction comprising oleate ester and the remaining alcohol, for example by filtration or centrifugation.

In respect to the fractionation of 2-monoolein, lower temperatures are required, for example, −20° C. In some embodiments of the present invention, intermediate purification b)ii) is performed by decreasing the temperature of the product mixture comprising 2-monoolein obtained in step b)i) to a temperature ranging from −30° C. to −10° C., for example to a temperature ranging from −25° C. to −15° C. or to a temperature ranging from −23° C. to −17° C. to allow fractionation via selective crystallization of 2-monoolein and by removing the remaining liquid fraction, for example by filtration.

Accordingly, fractionation temperatures below 0° C. of the crude mixtures and no addition of solvents allows for a simple and cheap purification step of 2-monoolein.

In some embodiments of the present invention, intermediate purification step b)ii) is performed by decreasing the temperature of the product mixture comprising 2-monoolein obtained in alcoholysis step b)i) to a temperature ranging from −25° C. to −15° C. to allow fractionation via selective precipitation of 2-monoolein and removing the remaining liquid fraction, for example by filtration or by centrifugation.

### Solvent-Free Esterification [steps a)iii) and b)iii)]

The process of the present invention comprises a process comprising the step of subjecting 2-monopalmitin deriving from step a)ii) to an esterification step under butanol and/or water removal conditions, in the presence of an immobilized lipase and of oleate ester and/or a mixture of fatty acids selected to allow the formation of 1,3-dioleo-2-palmitin (OPO) and/or of a customized profile of triacylglycerols comprising palmitic acid at sn-2 position and having a content of palmitic acid at sn-2 position which is equal or higher than 70% of total palmitic content.

The esterification step a)iii) is performed under butanol and/or water removal conditions, for example using nitrogen bubbling, molecular sieves or under vacuum (>10 mbar).

In some embodiments of the present invention, the esterification step a)iii) is performed under butanol and/or water removal conditions in presence of butyl oleate obtained in alcoholysis step b)i).

During the esterification step, the starting material, 2-monopalmitin, could undergo acyl migration leading to 1-monopalmitin. This is an unwanted conversion as it would ultimately lead to a lower sn-2 palmitate in the finish product. The inventors have found that performing the esterification step a)iii) in the presence of butyl oleate prevents and/or reduces the acyl migration, resulting in a more stable 2-monopalmitin at the temperatures needed for the reaction to run (>45° C. to have the 2-monopalmitin melted).

Solvent free enzymatic esterification of 2-monopalmitin to form OPO has been described in literature before. In the study Highly selective synthesis of 1,3-Oleoyl-2-Palmitoylglycerol by Lipase Catalysis (Schmid et al, 1999), OPO was synthesized using sn-1,3 specific lipases from Rhizomucor miehei and Rhizopus delemar immobilized on different carrier materials. The reaction was performed at 50° C. with 3 equivalence of oleic acid and highly purified 2-monopalmitin (through solvent crystallization at −25° C.). 10-25% immobilized lipase based on weight of 2-monopalmitin was used and the authors state 78% OPO was obtained with 96% sn-2 palmitic acid using Rhizopus delemar lipase immobilized on macroporous polypropylene (EP 100) after 16 h reaction. However, the same study revealed the limited temperature stability (at 52° C.) of such immobilized R. delemar lipase and, in addition, much longer reaction times were needed to reach high OPO concentrations during 2-monopalmitin esterification.

In pre-screening tests, pure 2-monopalmitin was used as starting material and three different immobilized lipases were evaluated; Lipozyme 435, Lipozyme TL IM, Novozymes 40145 NS. The most efficient lipases in forming TAGs were Novozymes NS 40 145 and TL IM. Lipozyme TL IM was then chosen as it had been proven to be the most effective in the alcoholysis reaction.

For the preparation of OPO or a mix of sn-2 palmitate TAG, oleic acid was replaced by butyl oleate, coming from the alcoholysis of triolein (step b)i)), or by a mixture of butyl oleate and free fatty acids (linoleic acid for instance). With an enzyme loading of 25% w/w immobilized lipase to 2-monopalmitin, the reaction was typically completed after 3 h.

Additionally, using the same lipase in both alcoholysis and esterification reaction steps makes the process more cost effective and allows re-use of the same immobilized enzyme preparation for both process steps a)i) and a)iii). The full process from tripalmitin enriched fat to OPO could be performed using only one lipase: Lipozyme TL IM.

The process of the present invention also comprises performing another process that is temporally and/or spatially separated from the other process comprising the step of subjecting 2-monoolein deriving from step b)ii) to an esterification step under butanol and/or water removal conditions, in the presence of an immobilized lipase and of oleate ester and/or a mixture of fatty acids selected to allow the formation of 2-Olein-1,3-dipalmitin (POP) and/or of a customized profile of triglycerides comprising oleic acid at sn-2 position at a level equal or higher than 70% of total oleic content.

The esterification step b)iii) is performed under butanol and/or water removal conditions, for example using nitrogen bubbling, molecular sieves or under vacuum (>10 mbar).

In some embodiments of the present invention, the esterification step b)iii) is performed under butanol and/or water removal conditions in presence of butyl palmitate obtained in alcoholysis step a)i).

For the preparation of POP or a mix of sn-2 oleate TAG, palmitic acid can be replaced by butyl palmitate, coming from the alcoholysis of tripalmitin, or by a mixture of butyl palmitate and free fatty acids (stearic acid for instance). With an enzyme loading of 25% w/w immobilized lipase to 2-monoolein, the reaction was typically completed after 3 h.

The process according to the present invention has then the advantage that the by-products generated in the 2-step processes involving the enzymatic reaction used to produce either OPO or POP can be recovered and re-used as reactants.

Accordingly, in some embodiments of the present invention, all or at least a portion of the palmitate ester generated in step a)i) and of the oleate ester generated in step b)i) is recycled in the respective processes after removal of the remaining alcohol by evaporation. By recycled, it is meant that the palmitate ester and the oleate ester are re-used as reactants in the respective reactions.

In some other embodiments, all or at least a portion of the butyl palmitate generated in step a)i) and of the butyl oleate generated in step b)i) is recycled in the respective processes after removal of the remaining alcohol by evaporation.

In alcoholysis steps a)i) and b)i), immobilized enzyme preparation allows to properly disperse the lipase in non-aqueous media, such as fats and solvents, and enables the recovery and reuse of the recovered enzyme in esterification steps a)iii) and b)iii), making the process more cost efficient.

In some embodiments of the present invention, esterification steps a)iii) and b)iii) are performed under butanol and/or water removal conditions at a temperature ranging from 35° C. to 60° C., for example at a temperature ranging from 40° C. to 50° C.

In some embodiments of the present invention, the esterification steps a)iii) and b)iii) are performed under butanol and/or water removal conditions at a temperature ranging from 35° C. to 60° C., for example at a temperature ranging from 40° C. to 50° C. in the presence of Thermomyces lanuginosis adsorbed on silica gel carrier.

### Purification [steps a)iv) and b)iv)]

The process of the present invention comprises a process comprising the step of purifying the product mixture obtained in step a)iii) to remove the excess of free fatty acids, remaining fatty acid alkyl esters and mono- and di-glycerides.

The process of the present invention also comprises performing another process that is temporally and/or spatially separated from the other process comprising the step of) purifying the product mixture obtained in step b)iii) to remove the excess of free fatty acids, remaining fatty acid alkyl esters and mono- and di-glycerides.

Purification of the final TAG product mixture deriving from esterification steps a)iii) and b)iii) according to the process of the present invention may be performed to remove the excess of free fatty acids, remaining fatty acid alkyl esters and mono-and di-glycerides.

The removal of the excess of free fatty acids, remaining fatty acid alkyl esters and mono- and di-glycerides may be performed using deodorization, distillation, fractionation or short-path distillation.

Typically, deodorization of the mixture and/or product that needs to be purified may be performed at a temperature higher than >200° C. and under vacuum conditions of pressure lower than 20 mBar.

### Process for the Preparation of Triacylglycerols Enriched in Oleic Acid at sn-2 Position

In one aspect of the invention, the present invention provides a process for the preparation of triacylglycerols enriched in oleic acid at sn-2 position comprising the steps of
a) subjecting triolein and/or triacylglycerols enriched in triolein to an alcoholysis step performed in the presence of an immobilized lipase and of a primary or secondary alcohol of a chain length C3-C5 to give a product mixture comprising 2-monoolein and oleate ester as a by-product;
b) purifying the mixture comprising 2-monoolein obtained in step a) by fractionation process via selective crystallization of 2-monoolein and subsequent removal of the remaining liquid fraction comprising oleate ester and the remaining alcohol, for example by filtration or by centrifugation;
c) subjecting 2-monoolein derived from step b) to an esterification step under butanol and/or water removal conditions, in the presence of an immobilized lipase and of butyl ester and/or a mixture of fatty acids selected to allow the formation POP (2-Olein-1,3-dipalmitin) and/or of a customized profile of triacylglycerols comprising oleic acid at sn-2 position and having a content of oleic acid in sn-2 position which is equal or higher than 70% of total oleic content; and
d) purifying the product mixture obtained in step c) to remove the excess of free fatty acids, remaining fatty acid alkyl esters and mono- and di-glycerides.

### Alcoholysis [step a)]

The process for the preparation of triacylglycerols enriched in oleic acid at sn-2 position comprises the step of subjecting triolein and/or triacylglycerols enriched in triolein to an alcoholysis step performed in the presence of an immobilized lipase and of a primary or secondary alcohol of a chain length C3-C5 to give a product mixture comprising 2-monoolein and oleate ester as a by-product.

In some embodiments of the present invention, the starting material for alcoholysis step a) is triolein. In other embodiments of the present invention, the starting material for alcoholysis step a) is a triacylglycerol mixture enriched in triolein, such as for example high oleic sunflower oil.

In some embodiments, the primary or secondary alcohol of chain length C3-C5 is selected from the list consisting of n-butanol, n-pentanol, isopropanol and mixture thereof.

In the context of the present invention, switching from the commonly used alcohols, methanol and ethanol, to n-butanol supplied at high molar ratio (6 to 15 equivalents) has surprisingly allowed the substrate to be solubilized at 50° C. without deactivating the enzyme, producing 2-monoolein with 90% purity.

In some embodiments of the present invention, the alcoholysis steps a) is performed in the presence of n-butanol, n-pentanol, isopropanol or mixtures thereof.

In some embodiments of the present invention, the alcoholysis step a) is performed in the presence of n-butanol. The n-butanol may be in excess.

By using n-butanol in alcoholysis step a), the reaction proceeded without any solvent at temperature ranging from 50 to 65° C. Butanol acts as both substrate and solubilization agent for the triacylglycerols, thereby, enabling a solvent-free reaction, high conversion yield and lipase activity.

In some embodiments, the by-product generated in alcoholysis step a) is butyl oleate. In another embodiment, triolein and/or triacylglycerols enriched in triolein is subjected to an alcoholysis step performed in the presence of an sn-1,3 lipase and of n-butanol to give a product mixture comprising 2-monoolein and butyl oleate as a by-product.

In some embodiments, alcoholysis step a) is performed in the presence of an sn-1,3 lipase selected in the group consisting of: lipase from Thermomyces lanuginosis adsorbed on silica (e.g., Lipozyme TL IM, Novozymes), lipase B from Candida antarctica adsorbed on methacrylate/divinylbenzene copolymer (e.g. Lipozyme 435, Novozymes) and lipase from Rhizomucor miehei attached via ion exchange on styrene/DVB polymer (e.g., Novozym® 40086, Novozymes) or via hydrophobic interaction onto macroporous polypropylene (Accurel EP 100).

In alcoholysis steps a), immobilized enzyme preparation allows to properly disperse the lipase in non-aqueous media, such as fats and solvents, and enables the recovery and re-use of the recovered enzyme in esterification steps b) making the process more cost efficient.

In some embodiments of the present invention, the alcoholysis step a) is performed in the presence of n-butanol and of a sn-1,3 lipase, for example from Thermomyces lanuginosis, adsorbed on silica gel carrier (e.g., Lipozyme TL IM, Novozymes). In other embodiments of the present invention, the alcoholysis step a) is performed in the presence of n-butanol and of Thermomyces lanuginosis adsorbed on silica gel carrier.

In another embodiment, triolein and/or triacylglycerols enriched in triolein is subjected to an alcoholysis step performed in the presence of an sn-1,3 lipase and of n-butanol to give a product mixture comprising 2-monoolein and butyl oleate as a by-product.

In some embodiments of the present invention, the alcoholysis step a) is performed at a temperature ranging from 40 to 70° C., for example at a temperature ranging from 45 to 55° C.

### Intermediate Purification [step b)]

The two-step enzymatic transesterification process according to the present invention is more complex than conventional methods of producing POP, e.g. single step acidolysis or interesterification, yet, the moderate increase in complexity enables to improve the quality in the final product significantly, i.e. higher sn-2 oleate and the possibility to access controlled mixtures of sn-2 oleate triacylglycerols, respectively, making it more attractive for use in in cocoa butter equivalents.

A two-step process requires the purification of the intermediate and it is important that the increase in quality is not offset by increase in cost potentially deriving from intermediate purification [steps b)].

Current technologies for intermediate purification include molecular distillation, solvent crystallization, and chromatography but all these three methods are too costly for the targeted application and would benefit from improvement/simplification. For example, solvent fractionation methods typically require solvent use and low temperatures (<−10° C.).

Accordingly, the process for the preparation of triacylglycerols enriched in oleic acid at sn-2 position comprises the step of purifying the mixture comprising 2-monoolein obtained in step a) by fractionation process via selective crystallization of 2-monoolein and subsequent removal of the remaining liquid fraction comprising oleate ester and the remaining alcohol, for example by filtration or centrifugation.

In respect to the fractionation of 2-monoolein, low temperatures are required, for example, −20° C. In some embodiments of the present invention, intermediate purification b) is performed by decreasing the temperature of the product mixture comprising 2-monoolein obtained in step a) to a temperature ranging from −30° C. to −10° C., for example to a temperature ranging from −25° C. to −15° C. or to a temperature ranging from −23° C. to −17° C. to allow fractionation via selective crystallization of 2-monoolein and by removing the remaining liquid fraction, for example by filtration or by centrifugation.

Accordingly, fractionation temperatures below 0° C. of the crude mixtures and no addition of solvents allows for a simple and cheap purification step of 2-monoolein.

In some embodiments of the present invention, intermediate purification step b) is performed by decreasing the temperature of the product mixture comprising 2-monoolein obtained in alcoholysis step a) to a temperature ranging from −25° C. to −15° C. to allow fractionation via selective precipitation of 2-monoolein and removing the remaining liquid fraction, for example by filtration or by centrifugation.

### Solvent-Free Esterification [step c)]

The process for the preparation of triacylglycerols enriched in oleic acid at sn-2 position further comprises subjecting 2-monoolein derived from step b) to an esterification step under butanol and/or water removal conditions in the presence of an immobilized lipase and of butyl ester and/or a mixture of fatty acids selected to allow the formation POP (2-Olein-1,3-dipalmitin) and/or of a customized profile of triacylglycerols comprising oleic acid at sn-2 position and having a content of oleic acid in sn-2 position which is equal or higher than 70% of total oleic content.

The esterification step c) is performed under butanol and/or water removal conditions, for example using nitrogen bubbling, molecular sieves or under vacuum (>10 mbar).

In some embodiments of the present invention, esterification step c) is performed under butanol and/or water removal conditions at a temperature ranging from 40° C. to 70° C., for example at a temperature ranging from 45° C. to 55° C.

In some embodiments of the present invention, the esterification step c) is performed under butanol and/or water removal conditions a temperature ranging from 35° C. to 60° C., for example at a temperature ranging from 40° C. to 50° C. in the presence of a lipase from Thermomyces lanuginosis adsorbed on silica gel carrier.

### Purification [step d)]

The process for the preparation of triacylglycerols enriched in oleic acid at sn-2 position further comprises purifying the product mixture obtained in step c) to remove the excess of free fatty acids, remaining fatty acid alkyl esters and mono-and di-glycerides.

Purification of the final TAG product mixture deriving from esterification step c) according to the process of the present invention may be performed to remove the excess of free fatty acids, remaining fatty acid alkyl esters and mono- and di-glycerides.

The removal of the excess of free fatty acids, remaining fatty acid alkyl esters and mono- and di-glycerides may be performed by deodorization, distillation, fractionation or short-path distillation.

Typically, deodorization of the mixture and/or product that needs to be purified may be performed at a temperature higher than >200° C. and under vacuum conditions of pressure lower than 20 mBar.

### Use of the Process for the Preparation of Triacylglycerols Enriched in Oleic Acid at sn-2 Position in a Process for the Preparation of Ingredients Comprising Triacylglycerols Enriched in Either Palmitic Acid at sn-2 Position or Oleic Acid at sn-2 Position

As previously mentioned, the by-product generated in the alcoholysis step of the process for producing POP, i.e. oleate ester, for example butyl oleate, can be used as reactant in the esterification step of the process to produce OPO, thereby resulting in a circular process. This circular process therefore provides all the advantages of the combined processes.

Accordingly, an aspect of the present invention provides the use of the process for the preparation of triacylglycerols enriched in oleic acid at sn-2 position in a process for the preparation of ingredients comprising triacylglycerols enriched in either palmitic acid at sn-2 position or in oleic acid at sn-2 position.

### EXPERIMENTAL SECTION

### Example 1

### Production of 2-Monopalmitin via Solvent-Free alcoholysis under Different Conditions

### Material and Methods

Alcoholysis was performed on pure tripalmitin in solvent free conditions using isopropanol, n-butanol or and n-pentanol as alcohols.

The study was performed to assess the viability of solvent free alcoholysis of tripalmitin to produce 2-monopalmitin, using alcohols of chain length C3-C5. For the process step to be viable, high conversions must be achieved to avoid the production of side products (e.g. diglycerides) that would impact the purification process and the reaction yield.

### Equipment

10×1.5 mL Agilent GC glass vials, screw-capped with septum
Thermomixer, with modified heating block to fit 1.5 mL Agilent GC-vials and temperature control

### Chemicals

Tripalmitin, Glycerol Tripalmitate, 98%, Alfa Aesar, LOT# 10184933
2-propanol, Honeywell, Chromasolv LC-MS
1-Butanol, Sigma-Aldrich, ≥99%
1-pentanol, Sigma-Aldrich, ≥99%

Alcohols were dried over molecular sieves (3 Å) prior to experiment.

### Enzymes

Lipozyme 435, Novozymes, Candida Antarctica lipase B immobilized on hydrophobic carrier (acryl resin)
Lipozyme TL IM, Novozymes, thermomyces lanuginosus lipase immobilized on silica gel carrier (non-compressible)

### Procedure

Thermomixer was heated to 50° C.
175 mg tripalmitin was weighed into 1.5 mL glass vials with tight screw caps containing a rubber septum for sampling
Alcohol was added to the vials and closed
The closed vials were placed in the thermomixer, shaken at 650 rpm until the substrate was fully dissolved
Prior to reaction start (0 min), a sample (10 μL) was taken
Reaction was started by adding the lipase
Samples were taken after 0, 30, 60, 120, 180 and 240 minutes

Table 1 below reports lipases and alcohols used in the experiment and mass and volume in each reaction vial. Duplicate mixes were prepared, making a total of 10 vials prepared and tested.

TABLE 1

| | | | Alcohol | | |
|---|---|---|---|---|---|
| Lipase | | | | | Equivalence |
| | Mass | | | Volume | (mol alcohol/ |
| Lipase | (mg) | % w/w | Alcohol | (μL) | mol tripalmitin) |
| Lipozyme | 15 | 12 | 2-propanol | 300 | 18 |
| 435 | | | | | |
| Lipozyme | 15 | 12 | n-butanol | 300 | 15 |
| 435 | | | | | |
| Lipozyme TL | 30 | 17 | 2-propanol | 300 | 18 |
| IM | | | | | |
| Lipozyme TL | 30 | 17 | n-butanol | 300 | 15 |
| IM | | | | | |
| Lipozyme TL | 30 | 17 | n-pentanol | 300 | 13 |
| IM | | | | | |

### Results and Discussion

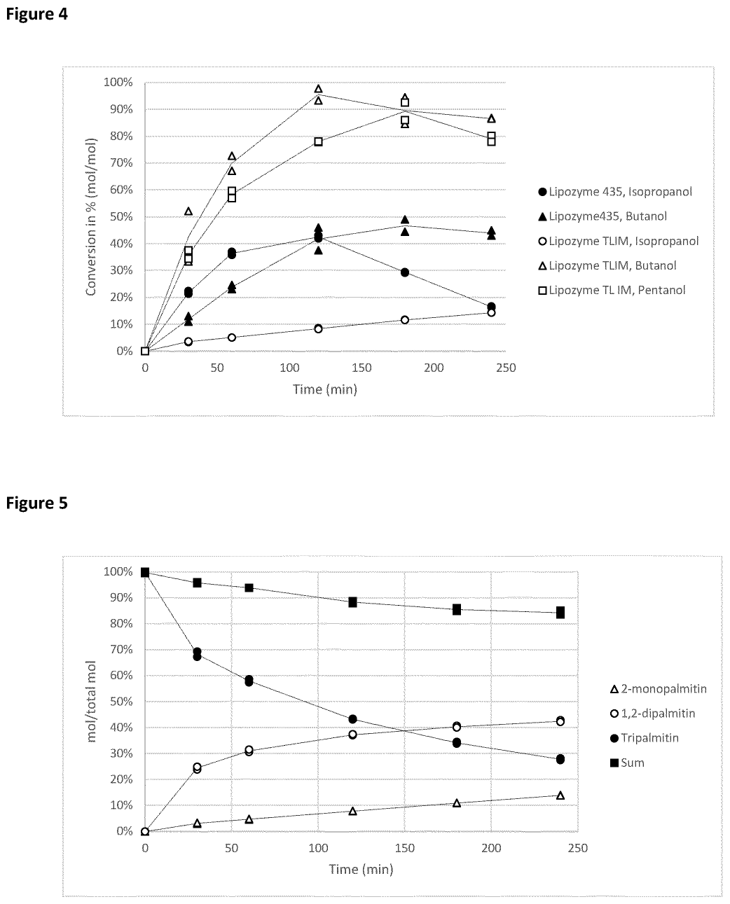

Results show that enzymatic alcoholysis of model substrate could be performed solvent free with alcohols of chain length C3-C5 using any of the lipase tested. The conversion yield of tripalmitin into 2-monopalmitin for each reaction were calculated for each sample point (and reported in FIG. 4). The best conversion yield achieved in the trial was 97%, using Lipozyme TL IM with n-butanol.

Tripalmitin was completely solubilized and miscible with the alcohols tested at 50° C. As a preliminary test, alcoholysis had been performed in ethanol, solvent-free. Because of the high melting point of tripalmitin, the reaction temperature needed to be increased to 65° C. to have a solubilized tripalmitin but under these conditions only low conversion of tripalmitin into 2-monopalmitin could be observed (33%, in the presence of Lipozyme 435, Novozymes). Attempting to dissolve tripalmitin at 50° C. by adding larger volumes of ethanol worked only poorly as the lipid and the alcohol were not fully miscible, giving a turbid suspension, and no enzymatic conversion was observed.

### Lipozyme TL IM

Higher yields were achieved using Lipozyme TL IM with the two alcohols: n-butanol and n-pentanol. The n-butanol reaction conversion reached its maximum after 2 h and the n-pentanol reaction after 3 h. The highest conversion achieved was with Lipozyme TL IM in n-butanol, reaching >95% after 2 h reaction. For Lipozyme TL IM, the reaction rates using isopropanol was lower than for the other two alcohols and the reaction didn't run to completion.

FIG. 5 shows the amount of tripalmitin, 1,2-dipalmitin and 2-monopalmitin expressed as molar fractions of the initial glyceride content. Shown is also the sum of the three fractions.

### Lipozyme 435

The highest conversion achieved using Lipozyme 435 was below 50% after 3 h reaction with n-butanol. With isopropanol, Lipozyme 435 achieved higher reaction rates than Lipozyme TL IM. The highest conversion achieved with isopropanol was 40%, reached after 2 h reaction with Lipozyme 435.

### Example 2

### Solvent-Free Butanolysis on a Fat High in Tripalmitin by Lipozyme TL IM

Alcoholysis of a fat rich in tripalmitin was performed to produce 2-monopalmitin in solvent-free conditions with an industrially relevant starting material.

The experiment confirmed that CristalGreen® (similarly to tripalmitin) may be a viable source of sn-2 palmitate for enzymatic production of 2-monopalmitin in reaction conditions using n-butanol and Lipozyme TL IM.

### Equipment

500 mL Schott flask with screw-cap equipped and tubing for gas sparging
Magnetic stirrer, stirrer plate
Water bath with heater/temperature control
2×100 mL Schott flasks with rubber lined screw-caps
Adolf Kühner Lab-Therm Lab shaker with temperature control

### Chemicals

1-Butanol, Sigma-Aldrich, ≥99%, dried over molecular sieves (3 Å)
CristalGreen®

### Enzymes

Lipozyme TL IM, Novozymes, thermomyces lanuginosus lipase immobilized on silica gel carrier (non-compressible)

### Procedure

### Drying CristalGreen®

100 g CristalGreen® was weighed into a 500 mL Schott flask
Flask was placed in a water bath at 70° C. and sparged with nitrogen gas for 6 h.

### Reaction (Duplicates)

To a 100 mL Schott flask was added:
   10 g dried CristalGreen®
   17 mL dry n-butanol
The flask was placed in a water bath at 70° C. until the fat was fully dissolved in butanol (clear, light yellow liquid)
The flask was placed in a Lab Shaker at 50° C. and 1400 rpm for 1 h
0 min sample was taken before reaction start (10 μL)
The reaction was started by adding 1.5 g Lipozyme TL IM
Samples were collected after 30, 60, 90, 120 and 150 minutes

### Lipase Reusability

High enzyme stability and reusability is one important driver for process economy and costs in enzymatic processes. Recyclability of Lipozyme TL IM was tested during alcoholysis by removing (filtration) the lipase after reaching full conversion, transferring it into a fresh substrate solution and then comparing the conversion yield and product profile for three consecutive reactions.

### Procedure

### Drying CristalGreen®

100 g CristalGreen® was weighed into a 500 mL Schott flask
Flask was placed in a water bath at 70° C. and sparged with nitrogen gas for 6 h.

Alcoholysis reaction was carried out in the same manner as described in Example 1 i.e. 10 g dried CristalGreen were reacted with 17 mL n-butanol using 1.5 g Lipozyme TL IM as biocatalyst. The reaction was carried out for 2.5 h before being stopped. Then the reaction was stopped by filtering off the enzyme. The same enzyme was then reused in an identical reaction for three cycles. It was shown that it was possible to re-use immobilized lipase TL in three alcoholysis reactions without losing its activity as similar product profiles were obtained for each reaction cycle.

### Results and Discussion

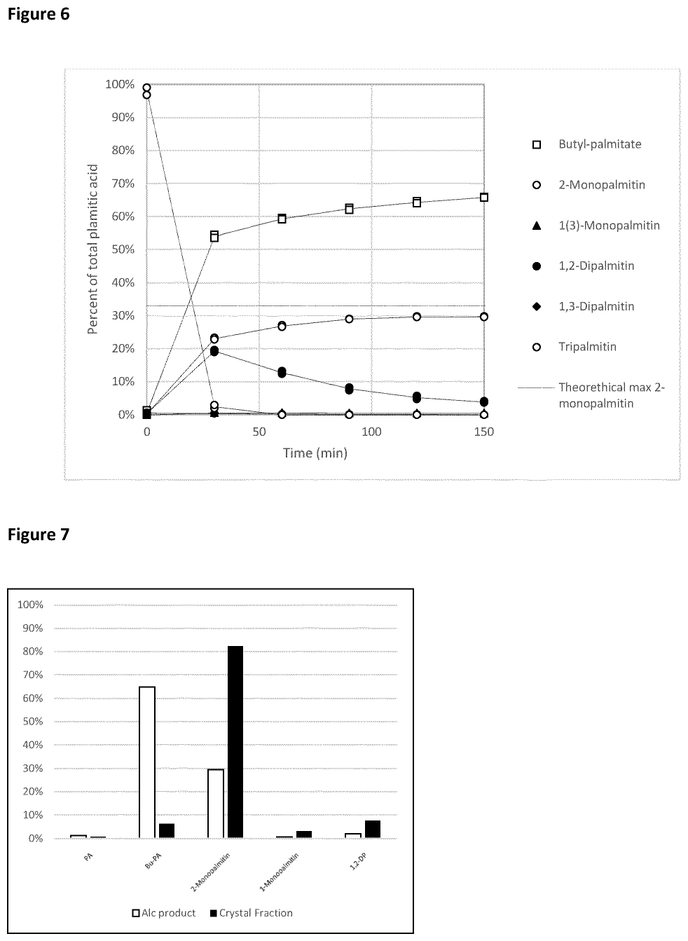

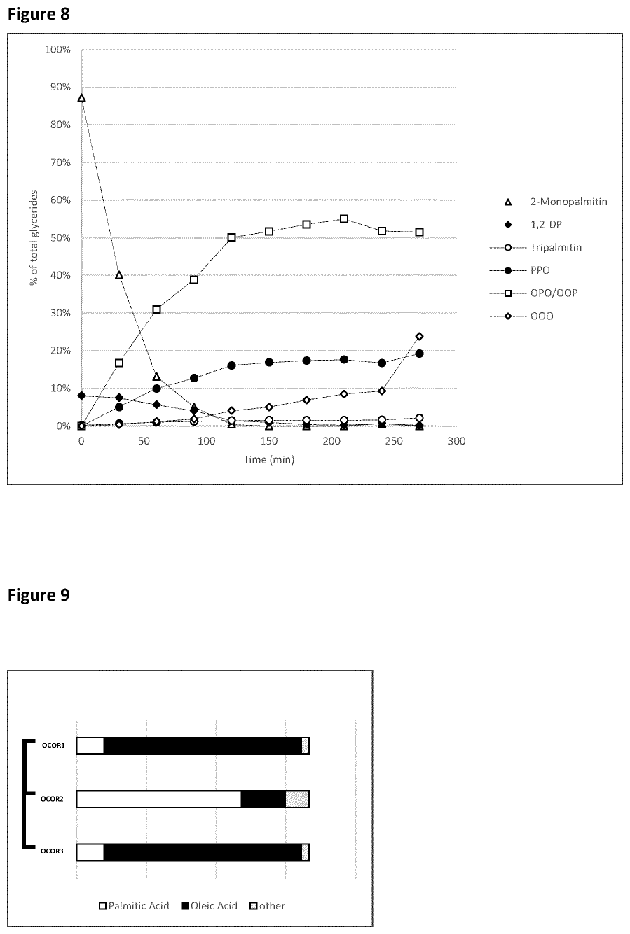

The reaction progress of the alcoholysis reaction with CristalGreen is shown in FIG. 6 and illustrates the depletion and formation of all species that contained palmitic acid (and were quantifiable by GC). The yield of 2-monopalmitin from CristalGreen® in this solvent-free alcoholysis reaction amounted to 94%, based on the palmitic acid content in Sn-2 position. The starting material Cristal Green contains 32% palmitic acid in Sn-2 position (the other palmitic acids located in Sn-1 and/or 3) and 30% palmitic acids were recovered in the final 2-monopalmitin product leading to a 94% yield. The remaining 6% of palmitic acids not present in Sn-2 position were found in the few side products, i.e., 1,2-diglycerides and free palmitic acid quantities. The palmitic acids originally present in Sn-1 and 3 of the starting material Cristal Green were converted into palmitic acid butyl ester.

### Example 3

### Study of Purification of 2-Monopalmitin by Solvent Free Fractionation (via Selective Precipitation)

2-monopalmitin was produced by n-butanolysis of CristalGreen® using Lipozyme TL IM as described in Example 2 and purified by solvent free fractionation via selective crystallization. To the 2-monopalmitin was added 2 equivalents of fatty acid alkyl ester and 13 equivalents of alcohol to create model mixtures for the study (as described below in Table 2). These mixes were then fractionated by gradually lowering the temperature in a water bath.

TABLE 2

| | | | | (2-MAG) | | | | |
|---|---|---|---|---|---|---|---|---|
| Palmitic acid | Weighed: | n: | n(2-MAG) | to weigh | n(alcohol) | m(alcohol) | V(alcohol) | V(alcohol) |
| Alkyl Ester | (mg) | (mmol) | (mol) | (mg): | (mol) | (mg) | (mL) | (μL) |
| Methyl- | 1058 | 3.91 | 1.96 | 645 | 0.0254 | 815 | 1.029 | 1029 |
| Ethyl- | 644 | 2.27 | 1.13 | 374 | 0.0147 | 679 | 0.860 | 860 |
| isopropyl- | 722 | 2.42 | 1.21 | 399 | 0.0157 | 946 | 1.203 | 1203 |
| n-butyl- | 600 | 1.92 | 0.96 | 317 | 0.0125 | 926 | 1.1444 | 1144 |
| n-pentyl- | 796 | 2.44 | 1.22 | 402 | 0.0159 | 1398 | 1.723 | 1723 |

### Part I—Preparing Palmitic Acid Alkyl Esters

Fatty acid alkyl esters were prepared from palmitic acid and alcohols methanol, ethanol, isopropanol, n-butanol and n-pentanol. The reaction was run in methyl-tert-butyl ether for the methanol and ethanol reactions. The other reactions were run solvent free. Lipozyme 435 catalyzed the reactions.

### Equipment

5×100 mL Schott flask with rubber lined screw-caps
Adolf Kühner Lab-Therm Lab shaker with temperature control
Büchi rotavapor—lab scale evaporator
Vacuum filtration setup
5×50 mL round flasks

One gram of palmitic acid was reacted using 1 gram of Lipozyme 435 in 10 mL alcohol for isopropanol, butanol, and pentanol. Methanol and ethanol preparations were performed with 1 mL alcohol and 10 ml MTBE. Molecular sieves (3 Å) were added to the mixtures for water removal.

The reaction was performed at 50° C. with a shaking of 1400 rpm. The reaction was started by adding the lipase and ran for 12 hours. The reaction was stopped by filtering off the lipase. After the reaction was stopped, the remaining alcohols and solvents were evaporated in a rotavapor.

The retained phase from the evaporation was transferred to clear 2 mL glass vials and weighed. The corresponding amounts of 2-monopalmitin and alcohol were calculated and added to the tubes as per Table 2.

### Part II—Crystallization/Fractionation Behavior of Mixtures of Fatty Acid Alkyl Ester, 2-Monopalmitin and Various Alcohols

The mixes prepared under Part I were placed in a water bath at 40° C. The temperature was then gradually lowered, and the phase transitions of the mixes and precipitation behavior were observed for the following 5 mixtures (as per Table 2):

methyl palmitate + 2-monopalmitate + methanol
ethyl palmitate + 2-monopalmitate + ethanol
isopropyl palmitate + 2-monopalmitate + isopropanol
n-butyl palmitate + 2-monopalmitin + n-butanol
n-pentyl palmitate + 2-monopalmitin + n-pentanol

| Melting points: | | |
|---|---|---|
| 2-monopalmitin: | 65° | C. |
| Methyl palmitate: | 30° | C. |
| Ethyl palmitate: | 24° | C. |
| n-propyl palmitate: | 20.4° | C. |
| n-butyl palmitate: | 16.9° | C. |

### Results and Discussion

As a result of the experiment, isopropyl-, n-butyl- and n-pentyl- mixtures could be fractionated, as 2-monopalmitin and 1,2-dipalmitin precipitated while the alcohol and its corresponding palmitic acid alkyl ester remained in solution. Methyl- and ethyl- mixes could not be fractionated.

The mixtures deriving from longer chain alcohols formed white crystals of 1,2-dipalmitin and 2-monopalmitin. The mixtures deriving from shorter chain could not be fractionated but rather the whole mix solidified.

From these results, it can be inferred that using a longer chain alcohol in the alcoholysis step aids in fractionation and makes solvent free fractionation possible.

Accordingly, having a C3-C5 alcohol gives the additional unexpected benefit of a simplified intermediate purification step for the desired product (2-monopalmitin).

### Example 4

### Intermediate Purification—Solvent-Free Fractionation via Selective Crystallization of Product Mixture Obtained by Solvent-Free Butanolysis of CristalGreen®

This study was performed to purify 2-monopalmitin from the product of the alcoholysis step as described in Example 1 via solvent free fractionation via selective precipitation.

### Equipment

50 mL Erlenmeyer flask
Vacuum filtration setup with glass filter

### Chemicals

From the alcoholysis step as described in Example 1, a final reaction mixture is obtained after 2.5 h reaction consisting of approximately 0.95 equivalence 2-monoglycerides, 0.05 eq. 1,2-diglycerides, 2 eq. fatty acid n-butyl esters, 13 eq. n-butanol
n-heptane

### Procedure

The alcoholysis reaction was stopped by filtering of the lipase
The filtrate was transferred to a 50 mL Erlenmeyer flask
The flask was placed at 4° C. overnight
Part of the fractionation mix was poured over the glass filter. The solution passes through, leaving a filter cake of white crystals. The crystals were washed by dripping heptane over them while still running the vacuum. The vacuum was then stopped, and the crystals scraped off the filter.
The crystals were dried in a desiccator and weighed.

Recovered from the fractionation and filtration was 1.62 g crystal fraction. The achieved overall process yield as described in Examples 2 and 4 was 40%.

### Result and Discussion

Intermediate purification by fractionation via selective crystallization of 2-monopalmitin was successfully performed on the final reaction mix from butanolysis of CristalGreen®. The amount of butyl palmitate was reduced by 90%. This shows that the method is viable for separating 2-monopalmitin (crystals) from liquid butyl palmitate and butanol, for example, via filtration.

### Example 5

### Solvent-Free Transesterification with Butyl Oleate of 2-Monopalmitin Product Derived from Butanolysis for OPO Ingredient Production

The present experiment was performed to demonstrate that 2-monopalmitin produced by butanolysis from CristalGreen® (as described in Example 2), purified by solvent-free fractionation via selective crystallization (as described in Example 4), can be successfully enzymatically transesterified with butyl oleate to produce OPO. The final ingredient contains a Sn-2 palmitate content matching that of human breast milk (70% or higher).

### Chemicals

Butyl oleate, Sigma-Aldrich
2-monopalmitin, produced through butanolysis from CristalGreen®, purified by solvent free fractionation via selective crystallization

### Enzymes

Lipozyme TL IM, Novozymes, Thermomyces lanuginosus lipase immobilized on silica gel carrier (non-compressible)

### Experiment

Added to a 25 mL flask:
   1.5 g 2-monopalmitin
   4 g butyl oleate (approx. 2.6 eq.)
The flask was placed in Rotary Evaporator at 50° C.
Stirring (by rotating the flask) and vacuum (˜20 mBar) were applied to dry the mixture before the addition of the enzymes
The reaction was started by addition of 375 mg of Lipozyme TL IM (25% w/w)
Stirring (by rotating the flask) at 50° C. under vacuum (20 mBar) until completion (˜4 h)
The reaction was stopped by removal of the immobilized enzymes by filtration
Excess of butyl oleate is removed by short-path distillation or deodorisation

As GC analysis method cannot distinguish between OPO and POO, further analysis using LC-MS was carried out on the final mixture showing OPO is the major TAG (54%) and sn-2 palmitate was 71%. The TAG distribution in the final TAG mixture is illustrated below in Table 3:

TABLE 3

| TAG | % |
|---|---|
| OPO | 54.5 |
| PPO | 22.5 |
| OOO | 9.8 |
| OOP | 3.6 |
| OSO | 2.6 |
| PPP | 2.1 |

### Result and Discussion

The esterification was successful which demonstrates that the enzyme can effectively use butyl oleate as a substrate to esterify oleic acid on 2-monopalmitin.

### Acyl Migration

During the esterification step, the starting material, 2-monopalmitin, could undergo acyl migration leading to 1-monopalmitin. This is an unwanted conversion as it would ultimately lead to a lower sn-2 palmitate in the finish product. We have conducted a stability test exposing 2-monopalmitin to 3 equivalents of either oleic acid or butyl oleate (without any enzyme) with increased temperatures every 1 h (1 h at 45° C. followed by 1 h at 50° C. followed by 1 h at 55° C.) to evaluate the acyl migration. Results of the GC analysis are shown below in Table 4:

TABLE 4

Ratio 2-monopalmitin/1-monopalmitin

| Temperature and time | With oleic acid | With butyl oleate |
|---|---|---|
| Initial T0 | 65 | 65 |
| After 45° C. for 1 h | 17.7 | 64.9 |
| then 50° C. for 1 h | 4.5 | 47 |
| then 55° C. for 1 h | 2.2 | 44.2 |

These results demonstrate that temperature is an important element to take into account in this reaction to prevent acyl migration as well as butyl oleate as the source of oleic acid over free oleic acid. Butyl oleate prevents acyl migration at 45° C. which is not the case for oleic acid.

### Example 6

### Solvent-Free Esterification with Butyl Oleate and Linoleic Acid of 2-Monopalmitin Product Derived from Butanolysis for OPO/OPL/LPL Ingredient Production

The present experiment was performed to demonstrate that 2-monopalmitin produced by butanolysis from CristalGreen® (as described in Example 2), purified by solvent-free fractionation via selective crystallization (as described in Example 4), can be successfully enzymatically esterified with a mixture a fatty acid ester together with a free fatty acid to produce a mixture of triglycerides enriched in palmitic acid at sn-2 position such as OPO, OPL and LPL. The final ingredient contains a Sn-2 palmitate content matching that of human breast milk (70% or higher).

### Chemicals

Butyl oleate, 99%, Sigma-Aldrich
Linoleic acid, 99%, Sigma-Aldrich
2-monopalmitin, produced through butanolysis from CristaiGreen®, purified by solvent free fractionation via selective crystallization

### Enzymes

Lipozyme TL IM, Novozymes, Thermomyces lanuginosus lipase immobilized on silica gel carrier (non-compressible)

### Experiment

Added to a 25 mL flask:
   1.5 g 2-monopalmitin
   3.07 g butyl oleate (2 eq.)
   2.55 g linoleic acid (2 eq.)
The flask was placed in Rotary Evaporator at 50° C.
Stirring (by rotating the flask) and vacuum (˜20 mBar) were applied to dry the mixture before the addition of the enzymes
The reaction was started by addition of 375 mg of Lipozyme TL IM (25% w/w)
Stirring (by rotating the flask) at 50° C. under vacuum (20 mBar) until completion (˜4 h)
The reaction was stopped by removal of the immobilized enzymes by filtration
Excess of butyl oleate and linoleic acid is removed by short-path distillation or deodorisation

As GC analysis method cannot distinguish between OPO and POO as well as between OPL, POL and OLP, further analysis using LC-MS was carried out on the final mixture showing OPL is the major TAG (31%) and sn-2 palmitate was 79%. The TAG distribution in the final TAG mixture is illustrated below in Table 5:

TABLE 5

| TAG | % |
|---|---|
| OPL | 31 |
| LPL | 17 |
| OPO | 15 |
| PPL | 7 |
| PPO | 6.3 |
| OOL | 3.9 |
| LOL | 2.3 |
| PLO | 2.3 |
| OOP | 1.8 |

### Result and Discussion

Level of LPL and OPO are very close (17% versus 15%) which might demonstrate a slight preference of the enzyme for the free fatty acid form versus the butyl ester one. It is also possible that the preference is coming from the fact that it is not the same fatty acid in the 2 forms (oleic vs linoleic).

The main conclusion of this example is that it is possible to mix the 2 different forms (free fatty acid and butyl ester) in the same reaction to introduce various fatty acids at the sn-1,3 positions.

### Example 7

### Solvent-Free Butanolysis on a Fat High in Triolein by Lipozyme TL IM

Alcoholysis of a fat rich in triolein (high oleic sunflower oil) was performed to produce 2-monoolein in solvent-free conditions with an industrially relevant starting material.

The experiment confirmed that high oleic sunflower oil may be a viable source of sn-2 oleate for enzymatic production of 2-monoolein in reaction conditions using n-butanol and Lipozyme TL IM.

### Chemicals

1-Butanol, 99%, Sigma-Aldrich
High oleic sunflower oil, Florin

### Enzymes

Lipozyme TL IM, Novozymes, thermomyces lanuginosus lipase immobilized on silica gel carrier (non-compressible)

### Procedure

### Reaction

To a 100 mL Schott flask was added:
   50 g high oleic sunflower oil (previously dried at 70° C. under vacuum)
   62 mL dry n-butanol
   7.5 g Lipozyme TL IM
The flask was placed in Rotary Evaporator at 50° C.
Samples were collected every hour for monitoring the reaction
After 4 h, no more TAG and only trace of DAG could be observed on the TLC
The reaction was stopped by filtrating the enzymes

### Example 8

### Intermediate Purification—Solvent-Free Fractionation via Selective Crystallization of Product Mixture Obtained by Solvent-Free Butanolysis of High Oleic Sunflower Oil

This study was performed to purify 2-monoolein from the product of the alcoholysis step as described in Example 7 via solvent free fractionation via selective precipitation.

### Chemicals

From the alcoholysis step as described in Example 7, a final reaction mixture is obtained after 2.5 h reaction consisting of 2-monoglycerides, 1,2-diglycerides, fatty acid n-butyl esters and n-butanol

### Procedure

The alcoholysis reaction was stopped by filtering of the lipase
The filtrate was transferred to a flask
The flask was cooled down slowly over night under slight stirring (rotation) up to −20° C.
A precipitate/crystallization was formed and was recovered after centrifugation and filtration at −20° C.
The crystals were collected, weighed and analyzed.
GC analysis showed together with 2-monoolein, traces of butyl oleate and DAG

Recovered from the fractionation and filtration was 10 g crystal fraction.

The achieved overall process yields as described in Examples 7 and 8 was 50%.

### Example 9

### Solvent-Free Transesterification with Butyl Palmitate of 2-Monoolein Product Derived from Butanolysis for POP Ingredient Production

The present experiment was performed to demonstrate that 2-monoolein produced by butanolysis from high oleic sunflower oil (as described in Example 7), purified by solvent-free fractionation via selective crystallization (as described in Example 8), can be successfully enzymatically transesterified with butyl palmitate to produce POP. The final ingredient contains an sn-2 oleate content of 70% or higher.

### Chemicals

Butyl palmitate, Sigma-Aldrich
2-monoolein, produced through butanolysis from high oleic sunflower oil, purified by solvent free fractionation via selective crystallization

### Enzymes

Lipozyme TL IM, Novozymes, Thermomyces lanuginosus lipase immobilized on silica gel carrier (non-compressible)

### Experiment

Added to a 25 mL flask:
   1 g 2-monoolein
   2.6 g butyl palmitate (3 eq.)
The flask was placed in Rotary Evaporator at 50° C.
Stirring (by rotating the flask) and vacuum (˜20 mBar) were applied to dry the mixture before the addition of the enzymes
The reaction was started by addition of 250 mg of Lipozyme TL IM (25% w/w)
Stirring (by rotating the flask) at 50° C. under vacuum (20 mBar) until completion (˜4 h)
The reaction was stopped by removal of the immobilized enzymes by filtration
Excess of butyl palmitate is removed by short-path distillation or deodorisation

### Result and Discussion

This example shows that it is possible to produce POP in an effective way. It is also possible to produce additional triglycerides in this esterification step by adding other fatty acid esters or free fatty acids (e.g. stearate ester or stearic acid) to even better match the TAG profile of cocoa butter. The final products can then be used as cocoa butter equivalents.

It should be understood that various changes and modifications to the presently preferred embodiments described herein will be apparent to those skilled in the art. Such changes and modifications can be made without departing from the spirit and scope of the present invention and without diminishing its attendant advantages. It is therefore intended that such changes and modifications be covered by the appended claims.

1. A process for the preparation of ingredients comprising triacylglycerols enriched either in palmitic acid at sn-2 position or in oleic acid at sn-2 position comprising:
a) performing one process comprising the steps of:
i) subjecting tripalmitin and/or triacylglycerols enriched in tripalmitin to an alcoholysis step in the presence of an immobilized lipase and of a primary or secondary alcohol of chain length C3-C5 to give a product mixture comprising 2-monopalmitin and palmitate ester as a by-product;
ii) purifying the product mixture comprising 2-monopalmitin obtained in alcoholysis step a)i) by fractionation process via selective crystallization of 2-monopalmitin and subsequent removal of the remaining liquid fraction comprising palmitate ester and the remaining alcohol;
iii) subjecting 2-monopalmitin deriving from step a)ii) to an esterification step under butanol and/or water removal conditions, in the presence of an immobilized lipase and of oleate ester and/or a mixture of fatty acids selected to allow the formation of 1,3-dioleo-2-palmitin (OPO) and/or of a customized profile of triacylglycerols comprising palmitic acid at sn-2 position and having a content of palmitic acid at sn-2 position which is equal or higher than 70% of total palmitic content;
iv) purifying the product mixture obtained in step a)iii) to remove the excess of free fatty acids, remaining fatty acid alkyl esters and mono- and di-glycerides;
b) performing another process, that is temporally and/or spatially separated from the process of step a), comprising the steps of:
i) subjecting triolein and/or triacylglycerols enriched in triolein to an alcoholysis step performed in the presence of an immobilized lipase and of a primary or secondary alcohol of a chain length C3-C5 to give a product mixture comprising 2-monoolein and oleate ester as a by-product;
ii) purifying the mixture comprising 2-monoolein obtained in alcoholysis step b)i) by fractionation process via selective crystallization of 2-monoolein and subsequent removal of the remaining liquid fraction comprising oleate ester and alcohol;
iii) subjecting 2-monoolein derived from step b)ii) to an esterification step under butanol and/or water removal conditions, in the presence of an immobilized lipase and of butyl ester and/or a mixture of fatty acids selected to allow the formation 2-Olein-1,3-dipalmitin (POP) and/or of a customized profile of triacylglycerol comprising oleic acid at sn-2 position and having a content of oleic acid at sn-2 position which is equal or higher than 70% of total oleic content;
iv) purifying the product mixture obtained in step b)iii) to remove the excess of free fatty acids, remaining fatty acid alkyl esters and mono- and di-glycerides; and
wherein all or at least a portion of the palmitate ester generated in step a)i) and of the oleate ester generated in step b)i) is recycled in the respective processes after removal of the remaining alcohol by evaporation.

2. A process according to claim 1 wherein the alcoholysis steps a)i) and b)i) are performed in the presence of n-butanol, n-pentanol, isopropanol or mixtures thereof.

3. A process according to claim 1 wherein alcoholysis steps a)i) and b)i) are performed in the presence of n-butanol and of an sn-1,3 lipase adsorbed on silica gel carrier.

4. A process according to claim 3 wherein the by-product generated in alcoholysis step a)i) is butyl palmitate and the by-product generated in alcoholysis step b)i) is butyl oleate.

5. A process according to claim 4 wherein the esterification step a)iii) is performed under butanol and/or water removal conditions in presence of butyl oleate obtained in alcoholysis step b)i).

6. A process according to claim 4 wherein the esterification step b)iii) is performed under butanol and/or water removal conditions in presence of butyl palmitate obtained in alcoholysis step a)i).

7. A process according to claim 1 wherein the starting material for alcoholysis step a)i) is a triacylglycerol mixture enriched in tripalmitin.

8. A process according to claim 1 wherein the starting material for alcoholysis step b)i) is a triacylglycerol mixture enriched in triolein.

9. A process according to claim 1 wherein alcoholysis steps a)i) and b)i) are performed at a temperature ranging from 40 to 70° C.

10. A process according to claim 1 wherein intermediate purification step a)ii) is performed by decreasing the temperature of the product mixture comprising 2-monopalmitate obtained in alcoholysis step a)i) to a temperature ranging from 0 to 15° C. to allow fractionation via selective crystallization of 2-monopalmitin and by removing the remaining liquid fraction.

11. A process according to claim 1 wherein intermediate purification step b)ii) is performed by decreasing the temperature of the product mixture comprising 2-monoolein obtained in alcoholysis step b)i) to a temperature ranging from −30° C. to −10 ° C. to allow fractionation via selective crystallization of 2-monoolein and by removing the remaining liquid fraction.

12. A process according to claim 1 wherein esterification steps a)iii) and b)iii) are performed under butanol and/or water removal conditions at a temperature ranging from 35° C. to 60° C. in the presence of Thermomyces lanuginosis adsorbed on silica gel carrier.

13. A process according to claim 1 wherein purification in steps a)iv) and b)iv) are performed using a process selected from the steps consisting of deodorization, distillation, fractionation and short-path distillation.

14. A process for the preparation of triacylglycerols enriched in oleic acid at sn-2 position comprising the steps of:
a) subjecting triolein and/or triacylglycerols enriched in triolein to an alcoholysis step performed in the presence of an immobilized lipase and of a primary or secondary alcohol of a chain length C3-C5 to give a product mixture comprising 2-monoolein and oleate ester as a by-product;
b) purifying the mixture comprising 2-monoolein obtained in step a) by fractionation process via selective crystallization of 2-monoolein and subsequent removal of the remaining liquid fraction comprising oleate ester and the remaining alcohol;
c) subjecting 2-monoolein derived from step b) to an esterification step under butanol and/or water removal conditions, in the presence of an immobilized lipase and of butyl ester and/or a mixture of fatty acids selected to allow the formation POP (2-Olein-1,3-dipalmitin) and/or of a customized profile of triacylglycerols comprising oleic acid at sn-2 position and having a content of oleic acid in sn-2 position which is equal or higher than 70% of total oleic content; and
d) purifying the product mixture obtained in step c) to remove the excess of free fatty acids, remaining fatty acid alkyl esters and mono- and di-glycerides.

15. A process according to claim 14 wherein alcoholysis of step a) is performed in the presence of n-butanol and of an sn-1,3 lipase adsorbed on silica gel carrier.

16. A process according to claim 15 wherein the by-product generated in alcoholysis step a) is butyl oleate.

17. (canceled)

18. A process according to claim 14 wherein the starting material for alcoholysis step a) is a triacylglycerol mixture enriched in triolein.

19. A process according to claim 14 wherein alcoholysis step a) is performed at a temperature ranging from 40 to 70° C.

20. A process according to claim 14 wherein intermediate purification step b) is performed by decreasing the temperature of the product mixture comprising 2-monoolein obtained in step a) to a temperature ranging from −30° C. to −10° C. to allow fractionation via selective crystallization of 2-monoolein and by removing the remaining liquid fraction, for example by filtration or by centrifugation.

21. A process according to claim 14 wherein esterification step c) is performed under butanol and/or water removal conditions at a temperature ranging from 35 to 60° C.

22-23. (canceled)
